(19) United States
(12) Patent Application Publication
(10) Pub. No.: US 2026/0097380 A1
(43) Pub. Date: Apr. 9, 2026

(54) PROCESS FOR THE PREPARATION OF MICROCAPSULES

(71) Applicant: Firmenich SA, Satigny (CH)

(72) Inventors: Damien Berthier, Satigny (CH); Geraldine Leon, Satigny (CH); Lahoussine Ouali, Satigny (CH)

(21) Appl. No.: 19/417,763

(22) Filed: Dec. 12, 2025

Related U.S. Application Data
(63) Continuation of application No. 16/954,229, now abandoned.

(30) Foreign Application Priority Data
Mar. 19, 2018 (EP) 18162589.8

(51) Int. Cl. B01J 13/16; A61K 8/11; A61K 8/73; A61K 8/84; A61Q 19/00; C08G 18/10; C08G 18/64; C08G 18/73; C11D 3/50; C11D 17/00

(52) U.S. Cl. CPC B01J 13/16; A61K 8/11; A61Q 19/00; C08G 18/10; C08G 18/6484; C08G 18/73; C11D 3/505; C11D 17/0039; A61K 8/732; A61K 8/736; A61K 8/84; A61K 2800/10; A61K 2800/56; A61K 2800/805

(57) ABSTRACT

Described herein are a new process for the preparation of microcapsules, and microcapsules obtainable by this process. Perfuming compositions and consumer products including the microcapsules, in particular perfumed consumer products in the form of home care or personal care products, are also described.

### CROSS-REFERENCE TO RELATED APPLICATIONS

This application is a continuation application of U.S. patent application Ser. No. 16/954,229, filed Jun. 16, 2020, which is a U.S. National Phase Application of International Patent Application No. PCT/EP19/56698, filed Mar. 18, 2019, which claims the benefit of priority to European Patent Application No. 18162589.8, filed Mar. 19, 2018, the entire contents of each of which are hereby incorporated by reference herein.

### TECHNICAL FIELD

The present invention relates to a new process for the preparation of core-shell microcapsules. Microcapsules obtainable by said process are also an object of the invention. Perfuming compositions and consumer products comprising said capsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

Polyurea and polyurethane-based microcapsule slurry are widely used for example in perfumery industry for instance as they provide a long lasting pleasant olfactory effect after their applications on different substrates. Those microcapsules have been widely disclosed in the prior art (see for example WO2007/004166 or EP 2300146 from the Applicant).

Therefore, there is still a need to use new microcapsules, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention provides a new process for the preparation of microcapsules, wherein a monomer reacts with modified starch during the interfacial polymerization in presence of chitosan.

### SUMMARY OF THE INVENTION

It has now been surprisingly found, that performing core-shell microcapsules encapsulating active ingredients could be obtained by reacting a monomer with modified starch in presence of chitosan during the interfacial polymerization. The process of the invention therefore provides a solution to the above-mentioned problems as it allows preparing microcapsules by using a limited amount of monomer during the interfacial polymerisation (for example polyisocyanates when preparing polyurea or polyurethane microcapsules). Unexpectedly, the applicant has found that the specific combination between chitosan and modified starch allowed obtaining microcapsules with the desired stability in challenging bases.

In a first aspect, the present invention relates to a process for preparing a core-shell microcapsule slurry, said process comprising the steps of:
(i) dissolving a monomer in an oil phase comprising a hydrophobic active ingredient, preferably a perfume;
(ii) preparing a dispersing phase comprising modified starch, wherein the dispersing phase is not miscible with the oil phase;
(iii) adding the oil phase to the dispersing phase to form a two-phases dispersion;
(iv) performing a curing step to form a microcapsule slurry;
characterized in that:
   chitosan is further added in the dispersing phase in step ii) and/or in the two-phases dispersion before performing step iv), and
   the weight ratio between chitosan and modified starch is comprised between 0.01 and 1.5.

In a second aspect, the invention relates to a core-shell microcapsule slurry obtainable by the process as defined above, wherein it comprises at least one microcapsule made of an oil-based core and a shell formed from the reaction between a monomer and modified starch in presence of chitosan.

A third object of the invention is a core-shell microcapsule slurry comprising at least one microcapsule made of:
an oil-based core; and
a shell comprising a copolymer, said copolymer comprising
   modified starch, preferably from 20 to 50% wt of modified starch;
   a monomer, preferably from 50 to 80% wt of a monomer; and
   chitosan, preferably greater than 0 to 20% wt of chitosan.

Another object of the invention is a copolymer comprising:
modified starch, preferably from 20 to 50% wt of modified starch;
a monomer, preferably from 50 to 80% wt of a monomer; and
chitosan, preferably greater than 0 to 20% wt of chitosan.

A perfuming composition comprising
(i) a microcapsule slurry as defined above, wherein the oil comprises a perfume;
(ii) at least one ingredient selected from the group consisting of a perfumery carrier and a perfumery co-ingredient; and
(iii) optionally at least one perfumery adjuvant is another object of this invention.

Consumer products comprising:
an active base; and
a microcapsule slurry or a perfuming composition as defined above,
   wherein the consumer product are in the form of a personal care composition or a home care composition respectively, are also part of the invention.

### DETAILED DESCRIPTION OF THE INVENTION

Unless stated otherwise, percentages (%) are meant to designate a percentage by weight of a composition.

By “active ingredient”, it is meant a single compound or a combination of ingredients. By “perfume or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles are dispersed in a continuous phase of a different composition and it specifically includes a suspension or an emulsion.

It has been found that core-shell microcapsules with overall good performance in terms of stability in a surfactant-based product and delivery of the active ingredient e.g. odor perception in the case of a perfume could be obtained when the monomer reacts with modified starch in presence of chitosan during the interfacial polymerization.

### Process for Preparing a Microcapsule Slurry

The present invention therefore relates in a first aspect to a process for preparing a core-shell microcapsule slurry, said process comprising the steps of:
(i) dissolving a monomer in an oil phase comprising a hydrophobic active ingredient, preferably a perfume;
(ii) preparing a dispersing phase comprising modified starch, wherein the dispersing phase is not miscible with the oil phase;
(iii) adding the oil phase to the dispersing phase to form a two-phases dispersion;
(iv) performing a curing step to form a microcapsule slurry;
   characterized in that:
chitosan is further added in the dispersing phase in step ii) and/or in the two-phases dispersion before performing step iv),
the weight ratio between chitosan and modified starch is comprised between 0.01 and 1.5.

In one step of the process, an oil phase is formed by admixing at least one hydrophobic active ingredient with at least one monomer.

By “monomer”, it is meant a molecule that, as unit, reacts or binds chemically to form a polymer or supramolecular polymer.

According to a particular embodiment, the monomer is not a polyepoxide.

According to an embodiment, the monomer is chosen in the group consisting of at least one polyisocyanate, poly anhydride, poly acyl chloride, acrylate monomers and polyalkoxysilane and mixtures thereof.

The monomer used in the process according to the invention is present in amounts representing from 0.1 to 15%, preferably from 0.5 to 8% and more preferably from 0.5 to 6% by weight based on the total weight of the oil phase.

According to a particular embodiment, the monomer is used in an amount between 0.1 and 4%, preferably between 0.1 and 2% by weight based on the total weight of the oil phase.

Indeed, it has been found that the reaction between the monomer and modified starch during the curing step (when the interfacial polymerization takes place) in presence of chitosan significantly decreases the amount of the monomer required during the process to provide a capsule wall with good properties.

Without being bound by any theory, it is believed that modified starch and chitosan react with the monomer in the shell.

According to a particular embodiment, the monomer added in step (i) is at least one polyisocyanate having at least two isocyanate functional groups.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the at least one polyisocyanate used in the process of the invention is present in amounts representing from 0.1 to 15%, preferably from 0.5 to 8% and more preferably from 0.5 to 6% by weight based on the total weight of the oil phase.

According to a particular embodiment, the monomer is used in an amount between 0.1 and 4%, preferably between 0.1 and 2% by weight based on the total weight of the oil phase.

Hydrophobic active ingredients used in the present invention are preferably chosen from the group consisting of flavor, flavor ingredients, perfume, perfume ingredients, nutraceuticals, cosmetics, insect control agents, biocide actives and mixtures thereof.

By “hydrophobic active ingredient”, it is meant any active ingredient-single ingredient or a mixture of ingredients-which forms a two-phases dispersion when mixed with a solvent, for example water.

According to a particular embodiment, the hydrophobic-active ingredient comprises a mixture of a perfume with another ingredient selected from the group consisting of nutraceuticals, cosmetics, insect control agents and biocide actives.

According to a particular embodiment, the hydrophobic active ingredient comprises a perfume.

According to a particular embodiment, the hydrophobic active ingredient consists of a perfume.

By “perfume” (or also “perfume oil”) what is meant here is an ingredient or composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odour. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodour counteraction, antimicrobial effect, microbial stability, insect control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate.

Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to any one of the invention's embodiments, the hydrophobic active ingredients represent between about 10% and 60% w/w, or even between 20% and 45% w/w, by weight, relative to the total weight of the dispersion as obtained after step iii).

According to a particular embodiment, the oil phase essentially consists of the polyisocyanate with at least 3 isocyanate functional groups, and a perfume or flavor oil.

In another step of the process according to the invention, modified starch is dissolved in a solvent to form a dispersing phase.

There is no restrictions regarding the nature of the solvent that can be used in step ii) as long as it can dissolve modified starch.

According to a particular embodiment, the dispersing phase consists of water.

According to another particular embodiment, the content of water is below or equal to 10%, preferably below or equal to 5%, more preferably below or equal to 3% by weight based on the total weight of the dispersing phase.

According to a particular embodiment, the dispersing phase is free of water.

According to an embodiment, the dispersing phase comprises a solvent chosen in the group consisting of glycerol, 1,4-butanediol, ethylene glycol and mixtures thereof.

Modified starch, also called starch derivatives, used in the present invention are prepared by physically, enzymatically, or chemically treating native starch to change its properties.

According to a particular embodiment, modified starch is chosen in the group consisting of modified food starch with octenylbutanedioate or starch sodium octenyl succinate, and mixtures thereof.

Modified starch is preferably comprised in an amount ranging from 0.1 to 5.0% by weight of the microcapsule slurry, preferably between 0.5 and 2 wt % of the microcapsule slurry.

In addition to modified starch, the dispersing phase can comprise at least one additional emulsifier, preferably chosen in the group consisting of carboxymethylated starch or cellulose.

Chitosan can be added directly in the dispersing phase before the emulsification and/or after the emulsification step before the curing step.

“Chitosan” and “N-acetylglucosamine polymer” are used indifferently in the present invention.

Preferably, chitosan is added in the form of a chitosan solution of acetic acid.

Preferably, chitosan is from non-animal origin.

According to the invention, the weight ratio between chitosan and modified starch is comprised between 0.01 and 1.5, preferably between 0.05 and 1.5, more preferably between 0.1 and 1.1, even more preferably between 0.15 and 0.5.

According to a particular embodiment, chitosan is added with modified starch in the dispersing phase.

In another step of the process of the invention, the oil phase is then added to the dispersing phase to form a two-phases dispersion (i.e an oil-in-water emulsion when the dispersing phase consists of water), wherein the mean droplet size is preferably comprised between 1 and 1000 □m, more preferably between 1 and 500 □m, and even more preferably between 5 and 50 microns.

The nature of the shell depends on the nature of the monomer present in the oil phase and the optional reactant present in the dispersing phase.

Thus, according to an embodiment, when the monomer is a polyisocyanate, microcapsules according to the present invention are polyurea-based capsules. According to this particular embodiment, interfacial polymerization is induced by addition of a polyamine reactant in the dispersing phase to form a polyurea wall with a polyisocyanate present in the oil phase. The amine is preferably chosen in the group consisting of guanidine salts, tris-(2-aminoethyl)amine, N,N,N′,N′-tetrakis(3-aminopropyl)-1,4-butanediamine, guanazole, aminoacids such as lysine, aminoalcohol such as 2-amino-1,3-propanediol, ethanolamine and mixtures thereof.

According to another embodiment, polyurea-based capsules are formed in absence of added polyamine reactant, and result only from the autopolymerization of the at least one polyisocyanate.

According to another embodiment, microcapsules according to the present invention are polyurethane-based capsules. According to this particular embodiment, the monomer is a polyisocyanate and interfacial polymerization is induced by the presence of a polyol in the dispersing phase.

Preferably the polyol reactant is selected from the group consisting of monomeric and polymeric polyols with multiple hydroxyl groups available for reaction and mixtures thereof.

According to another embodiment, capsules according to the present invention are polyurea/polyurethane based. In that case, the monomer is a polyisocyanate and interfacial polymerization is induced by addition of a mixture of the reactant mentioned under both precedent embodiments. Additionally, the monomer is a polyisocyanate, crosslinkers with both amino groups and hydroxyl groups can be used to generate polyurea/polyurethane materials. Furthermore, polyisocyanates with both urea and urethane functionalities can be used to generate polyurea/polyurethane materials.

As mentioned previously, chitosan can be added directly in the dispersing phase before the emulsification and/or after the emulsification step before the curing step.

Thus, according to an embodiment, the process comprises a further step of adding chitosan into the two-phases dispersion before step iv).

This is followed by a curing step iv) which allows ending up with microcapsules in the form of a slurry. According to a preferred embodiment, said step is performed at a temperature comprised between 6° and 80° C., possibly under pressure, for 1 to 4 hours. More preferably it is performed at between 5° and 90° C. for between 30 minutes and 4 hours.

According to the invention, the monomer reacts with modified starch in the presence of chitosan during the interfacial polymerisation (curing step) to form the microcapsules in form of a slurry.

According to a particular embodiment of the invention, at the end of step iv) one may also add to the invention's slurry a polymer selected from a non-ionic polysaccharide, a cationic polymer and mixtures thereof to form an outer coating to the microcapsules.

Non-ionic polysaccharide polymers are well known to a person skilled in the art and are described for instance in WO2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium 10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step iv). It is clearly understood by a person skilled in the art that only part of said added polymers will be incorporated into/deposited on the microcapsule shell.

Another object of the invention is a process for preparing a microcapsule powder comprising the steps as defined above and an additional step v) consisting of submitting the slurry obtained in step iv) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

### Microcapsule Slurry/Microcapsule Powder

Microcapsule slurry and microcapsule powder comprising at least one microcapsule made of an oil-based core and a shell formed from the reaction between a monomer as defined above and modified starch in presence of chitosan, obtainable by the processes above-described are also an object of the invention. Despite the low amount of monomer forming the membrane, the capsules of the invention show very good performance in terms of stability in challenging medium.

Microcapsules obtained by the process of the invention have a positive zeta potential, preferably comprised between +10 and +80 mV.

A suitable apparatus for measuring the zeta potential is Zetasizer Nano ZS (Malvern Instruments).

Another object of the invention is a core-shell microcapsule slurry comprising at least one microcapsule made of:
an oil-based core; and
a shell comprising a copolymer, said copolymer comprising
   modified starch, preferably from 20 to 50% wt of modified starch;
   a monomer, preferably from 50 to 80% wt of a monomer; and
   chitosan, preferably greater than 0 to 20% wt of chitosan.

The oil-based core comprises an hydrophobic active ingredient as described hereinabove.

Still another object of the invention is a copolymer comprising:
modified starch, preferably from 20 to 50% wt of modified starch;
a monomer, preferably from 50 to 80% wt of a monomer; and
chitosan, preferably greater than 0 to 20% wt of chitosan.

By “copolymer” it should be understood a polymer comprising more than one type of repeating unit.

According to an embodiment, the copolymer comprises between 0.1 and 20% wt of chitosan. The definitions such as oil-based core, core-shell microcapsule, modified starch, chitosan, monomer are the same as described hereinabove.

According to a particular embodiment, the monomer is a polyisocyanate having at least two isocyanate groups.

### Perfuming Composition/Consumer Products

Another object of the present invention is a perfuming composition comprising:
(i) a microcapsule slurry or a microcapsule powder as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.

As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.1 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

Another object of the invention is a consumer product comprising:
a personal care active base, and
a microcapsule slurry or a microcapsule powder as defined above or the perfuming composition as defined above,
wherein the consumer product is in the form of a personal care composition.

Another object of the invention is a consumer product comprising:
a home care or a fabric care active base, and
a microcapsule slurry or a microcapsule powder as defined above or the perfuming composition as defined above,
wherein the consumer product is in the form of a home care or a fabric care composition.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) microcapsule slurry as defined above,
d) optionally non-encapsulated perfume.

According to a particular embodiment, the consumer product as defined above is in a powder form and comprises:
(a) from 2 to 65% by weight, relative to the total weight of the consumer product, of at least one surfactant;
(b) microcapsule powder as defined above.
(c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer product can be a perfume, such as a fine perfume, a cologne, an after-shave lotion, a body-splash; a fabric care product, such as a liquid or solid detergent, tablets and pods, a fabric softener, a dryer sheet, a fabric refresher, an ironing water, or a bleach; a personal-care product, such as a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

According to a particular embodiment, the consumer product is in the form of a fabric softener composition and comprises:
between 85 and 99.9% of a fabric softener active base;
between 0.1 to 15 wt %, more preferably between 0.2 and 5 wt % by weight of the microcapsule slurry of the invention.

The fabric softener active base may comprise cationic surfactants of quaternary ammonium, such as Diethyl ester dimethyl ammonium chloride (DEEDMAC), TEAQ (triethanolamine quat), HEQ (Hamburg esterquat).

The invention will now be further described by way of examples. It will be appreciated that the invention as claimed is not intended to be limited in any way by these examples.

### EXAMPLES

### Example 1

### Preparation of Microcapsules According to the Invention with Chitosan (Post-Added), Modified Starch and Different Concentrations of Aromatic Polyisocyanate

### Microcapsules A-1

An aqueous solution of modified starch (42 g, 2 wt %, Gomme Purity 2000) was introduced in a beaker (pH=4.18). A solution of perfume oil A (see table 1, 25 g) and polyisocyanate (0.25 g, Takenate® D-110N, Origin: Mitsui Chemicals, Japan) was introduced into the beaker. The reaction mixture was stirrer at 24,000 rpm with an Ultra Turrax for 2 min at RT. A solution of chitosan in acetic acid 1 wt % in water (12 g, 2 wt %, Cs-G, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

TABLE 1
perfume oil A composition

| Raw material | wt % |
|---|---|
| Romascone ®^{a)} | 20 |
| Verdox ™^{b)} | 20 |
| Lorysia ®^{c)} | 20 |
| 3-(4-isopropylphenyl)-2-methylpropanal | 20 |
| Salicynile ®^{d)} | 20 |

^{a)}Methyl 2,2-dimethyl-6-methylene-1-cyclohexanecarboxylate, origin: Firmenich SA, Geneva, Switzerland
^{b)}2-tert-butyl-1-cyclohexyl acetate, trademark from International Flavors & Fragrances, USA
^{c)}4-(1,1-diméthyléthyl)-1-cyclohexyl acetate, origin: Firmenich SA, Geneva, Switzerland
^{d)}(2Z)-2-phenyl-2-hexenenitrile, origin: Firmenich SA, Geneva, Switzerland

### Microcapsules A-2

Microcapsules A-2 were prepared according to the protocol used to prepare capsule A-1 in the presence of chitosan Cs-H (12 g, 2 wt %, Origin: Kitozyme, Belgium).

### Microcapsules A-3 to A-20

Microcapsules A-3 to A-20 were prepared according to the protocol used to prepare capsule A-1 by using different amounts of Chitosan and polyisocyanate (see table 2).

TABLE 2
Microcapsules A-3 to A-20 composition

| Micro- | Polyisocyanate ¹⁾ | Chitosan Cs-G ²⁾ | Chitosan Cs-H ³⁾ |
|---|---|---|---|
| capsules | [g] | [g] | [g] |
| A-3 | 0.20 | 12 | |
| A-4 | 0.20 | | 12 |
| A-5 | 0.50 | 12 | |
| A-6 | 0.50 | | 12 |
| A-7 | 0.75 | 12 | |
| A-8 | 0.75 | | 12 |
| A-9 | 0.25 | 3 | |
| A-10 | 0.25 | | 3 |
| A-11 | 0.25 | 6 | |
| A-12 | 0.25 | | 6 |
| A-13 | 0.25 | 9 | |
| A-14 | 0.25 | | 9 |
| A-15 | 0.25 | 24 | |
| A-16 | 0.25 | | 24 |
| A-17 | 0.25 | 48 | |
| A-18 | 0.25 | | 48 |
| A-19 | 0.25 | 36 | |
| A-20 | 0.25 | | 36 |

¹⁾ Takenate ® D-110N, (75%)-trimethylol propane adduct of xylylene diisocyanate Origin: Mitsui Chemicals, Japan,
²⁾ Origin: Kitozyme, Belgium
³⁾ Origin: Kitozyme, Belgium

### Microcapsules A-21

An aqueous solution of modified starch (56 g, 2 wt %, Gomme Purity 2000) was introduced in a beaker (pH=4.18). A solution of perfume oil A (see table 1, 46.70 g) and polyisocyanate (0.47 g, Takenate® D-110N, Origin: Mitsui Chemicals, Japan) was introduced into the beaker. The reaction mixture was stirrer at 24,000 rpm with an Ultra Turrax for 2 min at RT. A solution of chitosan in acetic acid 1 wt % in water (67.20 g, 2 wt %, Cs-G, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

### Microcapsules A-22

An aqueous solution of modified starch (84 g, 2 wt %, Gomme Purity 2000) was introduced in a beaker (pH=4.18). A solution of perfume oil A (see table 1, 50 g) and polyisocyanate (0.50 g, Takenate® D-110N, Origin: Mitsui Chemicals, Japan) was introduced into the beaker. The reaction mixture was stirrer at 24,000 rpm with an Ultra Turrax for 2 min at RT. A solution of chitosan in acetic acid 1 wt % in water (60 g, 4 wt %, Cs-G, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

### Example 2

### Preparation of Microcapsules According to the Invention with Chitosan and Modified Starch (Simultaneous Addition) and Different Concentrations of Aromatic Polyisocyanate

### Microcapsules B-1

Aqueous solutions of modified starch (42 g, 2 wt %, Gomme Purity 2000) and chitosan in acetic acid 1 wt % in water (3 g, 2 wt %, Cs-G, Origin: Kitozyme, Belgium) were introduced in a beaker (pH=4.18). A solution of perfume oil (see table 1, 25 g) and polyisocyanate (0.25 g, Takenate® D-110N, Origin: Mitsui Chemicals, Japan) was introduced into the beaker. The reaction mixture was stirrer at 24,000 rpm with an Ultra Turrax for 2 min at RT. The resulting emulsion was then warmed up to 70° C. over the course of 1 h. Temperature was maintained at 70° C. for 2 h and then cooled down to RT to afford a white dispersion.

### Microcapsules B-2

Microcapsule B-2 were prepared according to the protocol used to prepare capsule B-1 in the presence of chitosan Cs-H (3 g, 2 wt %, Origin: Kitozyme, Belgium).

### Microcapsules B-3 to B-8

Microcapsules B-3 to B-8 were prepared according to the protocol used to prepare capsule B-1 by using different amounts of Chitosan and polyisocyanate (see table 3).

TABLE 3
Composition of microcapsules B-3 to B-8

| | | Chitosan Cs-G | Chitosan Cs-H |
|---|---|---|---|
| Microcapsules | Polyisocyanate [g] | [g] | [g] |
| B-3 | 0.25 | 6 | |
| B-4 | 0.25 | | 6 |
| B-5 | 0.25 | 9 | |
| B-6 | 0.25 | | 9 |
| B-7 | 0.25 | 12 | |
| B-8 | 0.25 | | 12 |

### Example 3

### Preparation of Microcapsules According to the Invention with Chitosan, Modified Starch and Aliphatic Polyisocyanate

### Microcapsules C1

An aqueous solution of modified starch (42 g, 2 wt %, Gomme Purity 2000,) was introduced in a beaker (pH=4.18). A solution of perfume oil (see Table 1, 25 g) and polyisocyanate (0.22 g, Desmodur® N-100, Origin: Covestro AG, Germany) was introduced into the beaker. The reaction mixture was stirrer at 24,000 rpm with an Ultra Turrax for 2 min at RT. An aqueous solution of chitosan (12 g, 2 wt %, Cs-G, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

### Microcapsules C2

An aqueous solution of modified starch (42 g, 2 wt %, Gomme Purity 2000) was introduced in a beaker (pH=4.18). A solution of perfume oil (see Table 1, 25 g) and polyisocyanate (0.22 g, Desmodur® N-100, Origin: Covestro AG, Germany) was introduced into the beaker. The reaction mixture was stirrer at 24,000 rpm with an Ultra Turrax for 2 min at RT. An aqueous solution of chitosan (12 g, 2 wt %, Cs-H, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

### Example 4

### Preparation of Microcapsules According to the Invention with Chitosan (Post-Added), Modified Starch and Aromatic Polyisocyanate and Comparative Microcapsule Free of Chitosan

### Microcapsules D1 to D14

An aqueous solution of modified starch (42 g, 2 wt %, Gomme Purity 2000) was introduced in a beaker (pH=4.18). A solution of perfume oil B (25 g)—see table 4 and polyisocyanate (0.25 g, Takenate® D-110N, Origin: Mitsui Chemicals, Japan) was introduced into the beaker. The reaction mixture was stirrer at 24,000 rpm with an Ultra Turrax for 2 min at RT. A solution of chitosan (see different amount in table 5, 2 wt % in acetic acid 1 wt % in water, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

TABLE 4
perfume oil B composition

| Raw Materials | % in oil |
|---|---|
| 2,4-Dimethyl-3-cyclohexene-1-carbaldehyde | 3.30% |
| Allyl Heptanoate | 5.50% |
| Allyl amyl glycolate | 10.99% |
| Delta Damascone | 1.65% |
| Verdyl acetate | 20.30% |
| Hedione ®¹⁾ | 4.95% |
| Iso E Super ®²⁾ | 16.49% |
| Ald. Hexylcinnamique | 9.89% |
| Ethyl-2-methylvalerate | 3.3% |
| Lilial | 21.98% |
| Pipol Butyrate | 1.1% |
| Ambrox ®³⁾ | 0.55% |
| Total | 100% |

¹⁾Methyl dihydrojasmonate, Firmenich SA, Geneva, Switzerland
²⁾1-(octahydro-2,3,8,8-tetramethyl-2-naphtalenyl)-1-ethanone, International Flavors & Fragrances, USA
³⁾(−)-(8R)-8,12-epoxy-13,14,15,16-tetranorlabdane, Firmenich SA, Geneva, Switzerland

TABLE 5
Composition of microcapsules D1 to D14

| | | Quantity | Zeta potential |
|---|---|---|---|
| Capsules | Chitosan | [g] | [mV] |
| D-1 | Cs-G | 12 | +35 |
| D-2 | Cs-H | 12 | +39 |
| D-3 | Cs-G | 9 | +25 |
| D-4 | Cs-H | 9 | +39 |
| D-5 | Cs-G | 6 | +19 |
| D-6 | Cs-H | 6 | +43 |
| D-7 | Cs-H | 15 | +47 |
| D-8 | Cs-H | 18 | +53 |
| D-9 | Cs-H | 21 | +45 |
| D-10 | Cs-H | 24 | +52 |
| D-11 | Cs-H | 34 | +56 |
| D-12 | Cs-G | 24 | +32 |
| D-13 | Cs-G | 36 | +30 |
| D-14 | No | 0 | −19 |
| (comparative) | | | |

### Microcapsules D-15

An aqueous solution of modified starch (30 g, 2 wt %, Gomme Purity 2000) was introduced in a beaker (pH=4.18). A solution of perfume oil B (see table 4, 25.00 g) and polyisocyanate (0.25 g, Takenate® D-110N, Origin: Mitsui Chemicals, Japan) was introduced into the beaker. The reaction mixture was stirrer at 24,000 rpm with an Ultra Turrax for 2 min at RT. A solution of chitosan in acetic acid 1 wt % in water (36.00 g, 2 wt %, Cs-H, Origin: Kitozyme, Belgium) was added dropwise with a syringe pump over the course of 1 h. The resulting emulsion was then warmed up to 70° C. over the course of 1 h. Temperature was maintained at 70° C. for 2 h and then cooled down to RT to afford a white dispersion.

### Example 5

### Preparation of Comparative Microcapsules

### Comparative Microcapsules E-1-PVOH as an Emulsifier and an Amine as a Cross-Linker

A solution of poly(vinyl alcohol) in water (45 g, 0.5 wt %, Mowiol 18-88, origin: Aldrich, Switzerland) was introduced in a beaker. A solution of perfume oil A (see table 1, 38 g) and polyisocyanate (0.27 g, Takenate® D-110N, Origin: Mitsui Chemicals, Japan) was introduced into the beaker. The reaction mixture was stirrer at 24,000 rpm with an Ultra Turrax for 2 min at room temperature (RT). A solution of guanidine carbonate (0.88 g, Origin: Aldrich, Switzerland) in water (4 g) was added dropwise with a syringe pump at room temperature over the course of 1 h. The resulting emulsion was warmed up to 70° C. over the course of 1 h. Temperature was maintained at 70° C. for 2 h and then cooled down to RT to afford a white dispersion (pH=9.7).

### Comparative Microcapsules E-2: Coacervate Chitosan/Gum Arabic

Solutions of gum Arabic (39.07 g, 16 wt % in water), chitosan (23.44 g, 4 wt % in aqueous solution acetic acid 1 wt %, Cs-G, Origin: Kitozyme, Belgium) and water (18.74 g) were introduced in a beaker. pH was adjusted to 1.7 with hydrogen chloride solution (1.1 g, 37 wt %, origin: Aldrich, Switzerland). Perfume oil A (62.5 g) was added and the reaction mixture was stirrer at 13,500 rpm with an Ultra Turrax for 2 min at RT to afford an emulsion. In a separated beaker, hydrogen chloride solution (0.98 g, 37 wt %) was added in water (300 mL, pH 1.7). Triethanolamine (38.7 g, solution at 5 wt % in water, Origin: Aldrich, Swtizerland) was added dropwise to obtain a pH at 2.78. Gluteraldehyde (2.5 g, 50 wt % in water, Origin: Aldrich, Switzerland) was added. The solution was added to the emulsion and the reaction mixture was stirred overnight. The capsule dispersion was then diluted with water (300 mL), filtered, washed with water (2500 mL) and filtered again to afford a dispersion (83.37 g). Dispersion was diluted in water (55 mL) and the pH was adjusted with Na₂CO₃ (0.23 g) to a value at 4.8 (total dispersion weight: 137.48 g).

### Comparative Microcapsules E-3-Free of Chitosan

An aqueous solution of modified starch (84 g, 2 wt %, Gomme Purity 2000) was introduced in a beaker (pH=4.18). A solution of perfume oil A (50 g) and polyisocyanate (0.50 g, Takenate® D-110N, Origin: Mitsui Chemicals, Japan) was introduced into the beaker. The reaction mixture was stirrer at 24,000 rpm with an Ultra Turrax for 2 min at RT. The resulting emulsion was stirred at room temperature for 1 h, then warmed up to 70° C. over the course of 1 h. Temperature was maintained at 70° C. for 2 h and then cooled down to RT to afford a white dispersion.

### Comparative Microcapsules E-4-Free of Modified Starch

A solution of chitosan (0.5 g, Cs-H, Mw=80,000 Da, origin: Kitozyme, Belgium) in an aqueous solution of acetic acid (1 wt %, 49.5 g) was introduced in a beaker (pH=4.02). A solution of perfume oil A (39 g) and polyisocyanate (0.50 g, Takenate® D-110N, Origin: Mitsui Chemicals, Japan) was introduced into the beaker. The reaction mixture was stirrer at 24,000 rpm with an Ultra Turrax for 2 min at RT. The resulting emulsion was warmed up to 70° C. over the course of 1 h. Temperature was maintained at 70° C. for 2 h and then cooled down to RT to afford a white dispersion.

### Comparative Microcapsules E-5-Free of Modified Starch

A solution of chitosan (Cs-G, Mw=15,000 Da, 2 g, origin: Kitozyme, Belgium) in an aqueous solution of acetic acid (1 wt %, 48 g) was introduced in a beaker (pH=4.78). A solution of perfume oil A (39 g) and polyisocyanate (0.27 g, Takenate® D-110N, Origin: Mitsui Chemicals, Japan) was introduced into the beaker. The reaction mixture was stirrer at 24,000 rpm with an Ultra Turrax for 2 min at room temperature (RT). The resulting emulsion was warmed up to 70° C. over the course of 1 h. Temperature was maintained at 70° C. for 2 h and then cooled down to RT to afford a white dispersion.

### Comparative Microcapsules E-6-Free of Modified Starch

A solution of chitosan (0.5 g, Cs-H, Mw=80,000 Da, origin: Kitozyme, Belgium) in an aqueous solution of acetic acid (1 wt %, 49.5 g) was introduced in a beaker (pH=4.02). A solution of perfume oil (see Table 1, 39 g) and polyisocyanate (0.27 g, Takenate® D-110N, Origin: Mitsui Chemicals, Japan) was introduced into the beaker. The reaction mixture was stirrer at 24,000 rpm with an Ultra Turrax for 2 min at RT. The resulting emulsion was warmed up to 70° C. over the course of 1 h. Temperature was maintained at 70° C. for 2 h and then cooled down to RT to afford a white dispersion.

### Comparative Microcapsules E-7-Free of Modified Starch

A solution of chitosan (Cs-G, Mw=15,000 Da, 2 g, origin: Kitozyme, Belgium) in an aqueous solution of acetic acid (1 wt %, 48 g) was introduced in a beaker (pH=4.78). A solution of perfume oil (see Table 1, 39 g) and polyisocyanate (0.50 g, Takenate® D-110N, Origin: Mitsui Chemicals, Japan) was introduced into the beaker. The reaction mixture was stirrer at 24,000 rpm with an Ultra Turrax for 2 min at room temperature (RT). The resulting emulsion was warmed up to 70° C. over the course of 1 h. Temperature was maintained at 70° C. for 2 h and then cooled down to RT to afford a white dispersion.

### Example 6

### Stability Performance in Aqueous Buffered Solutions

Capsules as defined in table 6 below were dispersed in aqueous buffered solutions at a perfume concentration of 0.5 wt %. Stability was measured in solutions at pH2, 4, 7 and 9 at RT for one month. Dispersion (circa 4 g) were mixed with a solution of 1,4-dibromobenzene in ethyl acetate at 150 ppm (10 mL). Quantity of lost perfume was determined by GC-FID.

TABLE 6
Perfume oil leakage (%)-1 month

| Microcapsules | pH 2 | pH 4 | pH 7 | pH 9 |
|---|---|---|---|---|
| Comparative | 29 | 27 | 25 | 39 |
| E-5 | | | | |
| Comparative | 10 | 12 | 8 | 21 |
| E-6 | | | | |
| A-1 | 1 | 1 | 1 | 1 |
| A-2 | 1 | 1 | 1 | 2 |
| A-3 | 4 | 4 | 8 | 6 |
| A-4 | 4 | 2 | 3 | 5 |
| B-8 | 1 | 0 | 2 | 4 |
| A-10 | 0 | 0 | 1 | 1 |
| A-14 | 2 | 1 | 3 | 5 |

One can conclude from those results that microcapsules prepared by the process of the invention show good stability in aqueous solution at different pH.

### Example 7

### Stability Performance in a Shower Gel

Capsules as defined in table 8 were dispersed in shower gel base described in table below to obtain a concentration of encapsulated perfume oil at 0.5%. Shower gel base and capsules were stored at RT for one month.

TABLE 7
Shower gel formulation

| Ingredients | % w/w |
|---|---|
| 1. Water deionised | 49.35 |
| 2. EDETA B Powder | 0.05 |
| Tetrasodium EDTA (Origin: BASF) | |
| 3. Carbopol ® Aqua SF-1 Polymer | 6.00 |
| Acrylates copolymer (Origin: Noveon) | |
| 4. Zetesol AO 328 U | 35.00 |
| Sodium C12-C15 Pareth Sulfate (Origin: Zschimmer & | |
| Schwarz) | |
| 5. Sodium hydroxide 20% aqueous solution | 1.00 |
| 6. Tego ® Betain F 50 | 8.00 |
| Cocamidopropyl Betaine (Origin: Goldschmidt AG) | |
| 7. Kathon CG | 0.10 |
| Methylchloroisothiazolinone and methylisothiazolinone | |
| (Origin: Rohm & Haas) | |
| 8. Citric acid 40% aqueous solution | 0.50 |

Shower gel (circa 4 g) were mixed with a solution of 1,4-dibromobenzene in ethyl acetate at 150 ppm (10 mL). Quantity of lost perfume was determined by GC-FID.

TABLE 8
Perfume oil leakage (%)-1 month

| Microcapsules | Oil leakage (%) |
|---|---|
| A-1 | 4 |
| A-2 | 5 |
| A-5 | 1 |
| A-6 | 1 |
| A-7 | 0 |
| A-8 | 0 |
| Comparative E-1 | 38 |
| Comparative E-2 | 27 |
| Comparative E-3 | 18 |
| Comparative E-4 | 68 |
| Comparative E-8 | 55 |
| Comparative E-9 | 50 |

One can conclude from those results that microcapsules prepared by the process of the invention (i.e comprising both modified starch and chitosan) show good stability after one month in a shower gel base compared to microcapsules outside the scope of the invention.

### Example 8

### Stability Performance in a Fabric Softener

The capsules of the present invention were tested in a fabric softening application using a fabric softener base with the following composition: Stepantex® VK90 (origin: Stepan) 16.5%, calcium chloride (10% in water) 0.6% and demineralized water 82.9%. Capsules were dispersed in fabric softener base at a concentration of encapsulated perfume oil of 0.5%. Fabric softener base and capsules were stored at RT for one month.

Capsules described above were dispersed in the fabric softener at a perfume concentration of 0.5 wt %. Softener (circa 4 g) were mixed with a solution of 1,4-dibromobenzene in ethyl acetate at 150 ppm (10 mL). Quantity of lost perfume was determined by GC-FID.

TABLE 9
Stability (1 month-RT)

| Microcapsules | Leakage (%) |
|---|---|
| Comparative E-6 | 47 |
| Comparative E-7 | 36 |
| A-1 | 9 |
| A-2 | 10 |
| A-4 | 16 |
| C-1 | 17 |
| B-8 | 15 |
| A-10 | 15 |

One can conclude from those results that microcapsules prepared by the process of the invention (i.e comprising both modified starch and chitosan) show good stability after one month in a fabric softener base compared to microcapsules outside the scope of the invention.

### Example 9

### Shell Composition of the Capsules of the Present Invention Determined by Elemental Analysis

### Elemental Analysis

Shell were extracted and analyzed by elemental analysis. Composition was estimated by calculation based on the component compositions.

| | | | | | Modified | | Takenate ® |
|---|---|---|---|---|---|---|---|
| | | | | | starch | Chitosan | D110N |
| Samples | % C | % H | % N | % O | [mol %] | [mol %] | [mol %] |
| Gomme | 41.3 | 6.4 | 0 | 52.3 | 100 | | |
| purity 2000 | | | | | | | |
| Chitosan Cs-G | 41.3 | 7.0 | 6.5 | 42.9 | | 100 | |
| Chitosan Cs-H | 42.0 | 7.0 | 7.6 | 42.3 | | 100 | |
| Takenate ® | 59.9 | 6.2 | 9.5 | 23.7 | | | 100 |
| D110N | | | | | | | |
| A-2 | 51.0 | 6.6 | 5.4 | 34.1 | 39.3 | 1.0 | 59.7 |
| A-10 | 53.9 | 6.4 | 6.9 | 29.7 | 23.9 | 0.3 | 75.8 |
| A-15 | 53.8 | 6.2 | 7.1 | 30.4 | 22.0 | 5.2 | 72.8 |
| A-16 | 54.2 | 6.2 | 7.4 | 29.8 | 19.3 | 6.1 | 74.6 |
| A-17 | 52.1 | 6.1 | 6.4 | 31 | 29.0 | 1.4 | 69.5 |
| A-18 | 53.2 | 6.1 | 7.3 | 30.6 | 19.8 | 10.3 | 69.9 |
| A-19 | 53.6 | 6.2 | 7 | 30.6 | 23.1 | 4.9 | 72.0 |
| A-20 | 54 | 6.2 | 7.3 | 30.6 | 20.7 | 7.4 | 71.9 |
| A-21 | 53.9 | 6.4 | 7 | 31.8 | 23.0 | 8.5 | 68.5 |
| D-15 | 53.7 | 6.3 | 7 | 31 | 23.7 | 5.5 | 70.8 |
| A-22 | 54 | 6.2 | 7.2 | 30.1 | 21.0 | 5.2 | 73.8 |

Elemental analyses have shown that the final shell composition comprises modified starch, chitosan and polyisocyanate.

1. A process for preparing a core-shell microcapsule slurry, said process comprising the steps of:
(i) dissolving a monomer in an oil phase comprising a hydrophobic active ingredient;
(ii) preparing a dispersing phase comprising modified starch, wherein the dispersing phase is not miscible with the oil phase;
(iii) adding the oil phase to the dispersing phase to form a two-phase dispersion; and
(iv) performing a curing step to form the core-shell microcapsule slurry;
wherein:
chitosan is further added in the dispersing phase in step ii) and/or in the two-phase dispersion before performing step iv),
a weight ratio between the chitosan and the modified starch is between 0.01 and 1.5,
the monomer reacts with the modified starch in the presence of chitosan during the curing step;
the process is conducted in the absence of polyamine such that a shell of the core-shell microcapsule slurry results only from autopolymerization of the at least one polyisocyanate; and
the monomer is used in an amount between 0.1 to 2% by weight based on a total weight of the oil phase.

2. The process according to claim 1, wherein the monomer is not a polyepoxide.

3. The process according to claim 1, wherein the dispersing phase is aqueous.

4. The process according to claim 1, wherein the monomer in the oil phase is selected from the group consisting of at least one polyisocyanate, polyanhydride, poly acyl chloride, acrylate monomers and polyalkoxysilane, and mixtures thereof.

5. The process according to claim 4, wherein the monomer is at least one polyisocyanate having at least two isocyanate groups.

6. The process according to claim 1, wherein the hydrophobic active ingredient comprises a perfume.

7. A core-shell microcapsule slurry obtainable by the process as defined in claim 1, wherein the core-shell microcapsule slurry comprises at least one microcapsule made of an oil-based core and a shell formed from the reaction between a monomer and modified starch in presence of chitosan.

8. A core-shell microcapsule slurry comprising at least one microcapsule made of:
an oil-based core; and
a shell comprising a copolymer, said copolymer comprising:
from 20 to 50% by weight of modified starch;
from 50 to 80% by weight of a monomer; and
greater than 0 to 20% by weight of chitosan.

9. A copolymer comprising:
from 20 to 50% by weight of modified starch;
from 50 to 80% by weight of a monomer; and
greater than 0 to 20% by weight of chitosan.

10. A perfuming composition comprising:
(i) a microcapsule slurry as defined in claim 7, wherein the hydrophobic active ingredient comprises a perfume;
(ii) at least one ingredient selected from the group consisting of a perfumery carrier and a perfuming co-ingredient; and
(iii) optionally a perfumery adjuvant.

11. A consumer product comprising:
a personal care active base; and
a microcapsule slurry as defined in claim 7,
wherein the consumer product is in the form of a personal care composition.

12. A consumer product comprising:
a home care or a fabric care active base; and
a microcapsule slurry as defined in claim 7,
wherein the consumer product is in the form of a home care or a fabric care composition.

13. The core-shell microcapsule slurry according to claim 8, wherein the monomer is a polyisocyanate having at least two isocyanate groups.

14. The copolymer according to claim 9, wherein the monomer is a polyisocyanate having at least two isocyanate groups.
